(19) United States
(12) Patent Application Publication
(10) Pub. No.: US 2008/0003686 A1
(43) Pub. Date: Jan. 3, 2008

(54) Prediction of relative polypeptide solubility by polyethylene glycol precipitation

(75) Inventors: Li Li, Sudbury, MA (US); Angela Kantor, Pepperell, MA (US); Nicholas W. Warne, Andover, MA (US)

Correspondence Address: WilmerHale/Wyeth, 60 STATE STREET, BOSTON, MA, 02109, US

(73) Assignee: Wyeth, Madison, NJ (US)

(21) Appl. No.: 11/804,191

(22) Filed: May 17, 2007

Related U.S. Application Data
(60) Provisional application No. 60/801,862, filed on May 19, 2006.

(51) Int. Cl. G01N 33/00

(52) U.S. Cl. 436/86

(57) ABSTRACT

A method is described for predicting the relative solubility of a polypeptide using polyethylene glycol (PEG) based volume exclusion precipitation. Different polypeptides can be tested for their solubilities relative to each other or relative to a reference. A single polypeptide can be tested for its relative solubility under different experimental conditions. The solubility determinations can be made by comparison based on graphs plotting the log solubility of the polypeptide against a range of PEG concentrations. Additionally, a method is provided for the high throughput visual or automated screening of multiple polypeptides for relative solubility differences, in a method that can omit the step of measuring the actual solubility or actual amount of precipitation of each sample at each PEG concentration.

### CROSS-REFERENCE TO RELATED APPLICATIONS

This application claims priority to provisional U.S. Application Ser. No. 60/801,862, filed on May 19, 2006, which is herein incorporated by reference in its entirety.

### FIELD OF THE INVENTION

The present invention relates generally to the field of protein characterization. More specifically, the invention relates to methods of predicting protein solubility.

### BACKGROUND OF THE INVENTION

An important aspect of formulating pharmaceutical compositions that contain a polypeptide is determining the solubility of the polypeptide to be used in a preparation. Procedures for determining solubility are generally not easily used for evaluating the solubility of large numbers of polypeptides because, for example, of the number of manipulations used and difficulties with obtaining large enough quantities of the polypeptide(s) to be tested.

Polyethylene glycol (PEG) is a non-toxic, non-adsorbing, synthetic long-chain amphiphilic polymer that is widely used in a number of industrial applications. PEG is a useful molecule within a laboratory or industrial setting because it can be used at ambient temperatures for polypeptide precipitation.

There is a need for a high throughput screening method to assay the solubility of polypeptides that are candidates, e.g., as drugs, at an early stage of discovery or development, and thereby to identify those polypeptides that may possess problematic solubility at a relatively early stage of development, for example, before commercial scaling. Additionally, minimizing the amount of starting material required for testing solubility is advantageous, e.g., when the polypeptide is available only in very limited amounts.

### SUMMARY OF THE INVENTION

The invention relates to methods for predicting the relative solubility of one or more polypeptides comprising precipitating the polypeptides using PEG volume exclusion. The assay is referred to herein as a “relative solubility assay” or “PEG precipitation assay.” More particularly, the test polypeptides assayed by the present method can be compared to one or more polypeptides of known solubility to detect those polypeptides with potentially difficult solubility problems prior to the time-consuming and expensive commercial scale-up of producing the test polypeptide. The method can also be used to identify parameters suitable for various uses of a selected polypeptide.

Accordingly, the invention relates to a method for predicting the relative solubility of a test polypeptide. The method includes providing one or more samples of a test polypeptide in a solution, thereby providing test samples; contacting the test samples with different concentrations of polyethylene glycol (PEG) thereby forming a precipitated sample; determining the precipitation of each test sample contacted with PEG; and correlating the amount of precipitation of the test polypeptide in the precipitated sample with solubility of at least one reference polypeptide sample analyzed under corresponding conditions, thereby determining the solubility of the test polypeptide relative to the reference polypeptide sample; or correlating the amount of precipitation of the test polypeptide in the precipitated sample(s) under different experimental conditions, thereby determining the relative solubility of the test polypeptide under each experimental condition. In some embodiments, the test polypeptide is an antibody or a fragment of an antibody, a molecule that can bind to a ligand, or a soluble receptor. In certain embodiments, the method also includes graphing the log of the solubility values determined for each sample against the PEG concentration of that sample and extrapolating the resulting line to zero percent PEG, thereby providing an apparent solubility value for the polypeptide. In some cases, the test polypeptide does not bind to PEG. In certain embodiments, the PEG precipitation of a test polypeptide is reversible. The PEG precipitation may, in some cases, not change the secondary structure of the test polypeptide. For some embodiments, the starting concentration of the test polypeptide to be analyzed does not substantially affect the resulting solubility value. The method also includes embodiments in which increasing the temperature increases the solubility value for a selected PEG concentration or the addition of sucrose to the buffer increases the solubility of the test polypeptide. The method also can be practiced such that the slope of the curve resulting from plotting the log solubility values of a higher molecular weight polypeptide sample against the PEG concentration increases relative to the slope of the curve of a lower molecular weight polypeptide. In some embodiments of the invention, the reference is a polypeptide of known solubility. In some cases, several polypeptides of known solubility are used as references, e.g., to establish a standard curve with which the relative solubility of a test polypeptide can be determined. In certain cases, the reference polypeptide(s) are selected to be of a similar type to the test polypeptide, for example, antibodies of known solubility can be used as reference polypeptides when determining the relative solubility of test polypeptides that are antibodies. In some embodiments, precipitation is assayed by determining turbidity of the precipitated sample(s). In some embodiments, the precipitated sample is centrifuged and the amount of precipitate is determined, the amount of protein in the supernatant is determined, or the amount of protein in the precipitate is determined.

In another aspect, the invention relates to a method for determining the relative solubility of a polypeptide compared to at least one other polypeptide of approximately the same molecular weight. The method includes providing a sample of at least two different polypeptides at the same concentration; contacting each polypeptide sample with a range of test PEG concentrations; determining the lowest test PEG concentration that precipitates a polypeptide sample, thereby determining a minimum percentage of PEG that precipitates each polypeptide; and correlating the minimum percentage of PEG with the solubility of each polypeptide relative to each other polypeptide. In some embodiments of the method, one or more manipulations of the assay are performed in a 96-well plate format. In some embodiments of the method, the range of PEG concentrations is about 2%-16%. The plate or other multisample format may be read visually by determining the smallest test concentration of PEG that causes opalescence of a sample. In some cases, the opalescence of samples in the plate is read using an automated plate reader.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

Other features and advantages of the invention will be apparent from the detailed description, drawings, and from the claims.

### BRIEF DESCRIPTION OF THE DRAWINGS

FIG. 1 is a graph depicting the results of a binding study of P1 with PEG-10K by Fourier Transform Infrared Spectrometry (FTIR).

FIG. 2 is a graph depicting the results of a secondary structure analysis of P1 by FTIR.

FIG. 3 is a bar graph depicting the results of an experiment designed to test whether polypeptide precipitation with PEG is fully reversible.

FIG. 4 is a graph depicting the results of experiments comparing the accuracy of solubility prediction by PEG-10K and PEG-20K. Solubility was tested using PEG-10K and PEG-20K to compare the effectiveness of volume-exclusion methodology with alternative molecular weight PEG.

FIG. 5A is a graph depicting the results of experiments in which polypeptides with different molecular weights were used to test the effect of polypeptide size on the phase diagram.

FIG. 5B is a graph depicting the relationship between molecular weight of a polypeptide and the slope of the line in a graph (as in FIGS. 1 and 2) representing solubility versus PEG precipitation percentage.

FIG. 6A is a graph indicating the reproducibility of polypeptide solubility prediction for P4. The experiments were performed in triplicate. 20 mM succinate is the formulation buffer for P4.

FIG. 6B is a graph indicating the reproducibility of polypeptide solubility prediction for P1. The experiments were performed in triplicate. 50 mM histidine is the formulation buffer for P1.

FIG. 7A is a graph depicting the effect of polypeptide concentration on the PEG-determined solubility of P4 in 20 mM succinate pH6.0. Initial polypeptide concentrations are 5.5 mg/mL (squares) and 11 mg/mL (diamonds).

FIG. 7B is a graph depicting the effect of polypeptide concentration on the PEG-determined solubility of P1 in 20 mM succinate pH6.0. Initial polypeptide concentrations are 5.5 mg/mL (squares) and 11 mg/mL (diamonds).

FIG. 8A is a graph depicting the effect of variable temperature (diamonds, 20° C. or squares, 0° C.) on predicted solubility of P4 in 20 mM succinate, pH 6.0.

FIG. 8B is a graph depicting the effect of variable temperature (diamonds, 20° C. or triangles, 0° C.) on predicted solubility of P1 in 20 mM succinate, pH 6.0.

FIG. 9 is a graph illustrating the effect of pH (triangles, 20 mM succinate, pH 6.0; squares, 10 mM phosphate, pH 7.0; diamonds 10 mM Tris, pH 8.0) on solubility estimation of P1.

FIG. 10 is a graph of the pH profile of P1 solubility predicted by PEG-10K at 0° C. and 20° C.

FIG. 11 is a graph illustrating the effect of the ionic strength of the buffer on the performance of PEG precipitation method using P1 at 10 mg/mL.

FIG. 12A is a graph depicting the results of experiments assaying the effect of sucrose on P5 apparent solubility with NaCl added to the PEG-precipitation buffer.

FIG. 12B is a graph depicting the results of experiments assaying the effect of sucrose on P5 apparent solubility without NaCl added to the PEG-precipitation buffer.

FIG. 13 is a reproduction of a photograph of 96-well plates used in high throughput screening (HTS) to determine the apparent solubility of monoclonal antibody using a PEG precipitation method.

FIG. 14 is a graph depicting the correlation of opalescence of monoclonal antibody solutions at a concentration of 90 mg/mL with relative solubility predicted by the PEG precipitation method.

### DETAILED DESCRIPTION OF THE INVENTION

The methods disclosed herein provide advantages for evaluation of polypeptide characteristics, e.g., solubility. The methods assay the relative solubilities of polypeptides such as antibodies or fragments of antibodies, using a limited number of manipulations. Limiting the number of manipulations is an advantage, for example, because it can reduce the amount of time to obtain a solubility measurement for a polypeptide or group of polypeptides, and because fewer manipulations minimizes the amount of polypeptide lost in processing.

Relative Solubility Assay

The invention relates to the need for a relatively rapid and efficient method for estimating the relative solubility of a polypeptide (a relative solubility assay). In general, the method employs PEG precipitation in a method for assaying relative solubility, which can decrease the amount of starting polypeptide for a solubility assay from approximately 200 mg in conventional approaches that measure actual solubility using a membrane-based concentration approach, to about 10 mg to about 30 mg (e.g., about 5 mg to about 100 mg, about 5 mg to about 50 mg, or about 10 mg to about 50 mg). The assay method does not preclude the use of larger amounts of polypeptide.

In some embodiments, the assay includes adding selected concentrations of PEG (a PEG precipitation series) to test samples containing a polypeptide of interest in solution (test polypeptide; a selected protein), determining the saturation concentration of the polypeptide at each PEG concentration, and comparing the extrapolated value of the saturation line at zero PEG concentration with at least one additional (i.e., different) polypeptide tested under the same assay conditions. In other embodiments, a test polypeptide is prepared under two or more different conditions such as different buffer components, pH, or temperature and tested for solubility with varying PEG concentrations. Saturated concentration, which is obtained by measuring polypeptide concentration in the supernatant of samples in which precipitation is observed, can be plotted in log scale against corresponding PEG concentration. The Y-intercept of the fitted line provides the apparent solubility of the polypeptide at zero PEG, and the slope of the line can be also calculated. Although the apparent solubility can be very different from actual achievable solubility determined using a membrane-based concentration approach, the apparent solubility can be utilized to compare relative solubility of one polypeptide to another. The slope of the fitted line is related to the molecular sizes of PEG and polypeptide, while it is unrelated to pH, temperature, and buffer.

In one embodiment, the invention provides a method for predicting the relative solubility of a polypeptide (e.g., a test polypeptide), the method comprising providing at least one sample of a test polypeptide in a solution, contacting each sample of the test polypeptide with a different concentration of polyethylene glycol (PEG), determining the relative solubility (e.g., by testing the amount of precipitation) of each sample at a given PEG concentration, and comparing the solubility of the test polypeptide to the solubility of a reference polypeptide sample or second test polypeptide sample analyzed under corresponding conditions, thereby determining the relative solubility of the test polypeptide compared to the reference or second test polypeptide. Additional test polypeptides may be tested for relative solubility, e.g., three, four, five, ten, twenty, fifty, one hundred, one thousand, or more, using the method. In some cases, the relative solubility of multiple samples of the test polypeptide prepared or tested under different experimental conditions is compared, thereby determining the solubility of the test polypeptide relative to the second polypeptide or set of experimental conditions. In certain embodiments, the polypeptides are proteins, e.g., antibodies, antibody fragments, ligand-binding molecules, or soluble receptors. More than one type of polypeptide can be used in an assay or the assay may utilize polypeptides that are all of the same or similar type, e.g., all antibodies.

The invention further relates to a method as described herein that also includes graphing the log of the solubility values determined for each sample against the PEG concentration of that sample and extrapolating the resulting line to zero percent PEG, thereby providing an apparent solubility value for a given polypeptide sample, or a set of solubility values for the tested polypeptides. In some aspects of the method, the polypeptide does not bind to PEG, the PEG precipitation is reversible, the PEG does not change the secondary structure of the polypeptide, or the starting concentration of the polypeptide to be analyzed does not substantially affect the resulting solubility value. Further aspects of the method include increasing the temperature to increase the solubility value for a given (selected) PEG concentration, or adding sucrose to the buffer to affect (e.g., increase) the solubility of the polypeptide.

In still another aspect, the method for predicting the relative solubility of a polypeptide is performed and analyzed such that the slope of the curve resulting from plotting the log solubility values of a higher molecular weight polypeptide sample against the PEG concentration increases relative to the slope of the curve of a lower molecular weight polypeptide sample.

In another embodiment, the method provided herein can also include providing multiple polypeptide samples of different polypeptides at the same concentration and each different polypeptide is mixed with a range of PEG concentrations, the minimum percentage of PEG (that is, the minimum percentage of a tested PEG concentration) that precipitates each different polypeptide is determined (the minimum precipitating PEG concentration, MPPC, which can be expressed as a percentage or concentration), and MPPC is correlated with the solubility of the polypeptide relative to the other polypeptide samples.

In some embodiments, the polypeptide samples used in a method described herein are analyzed in a 96-well plate format. In general, the range of PEG concentrations is about 2-16%. The plate can be read visually by determining the smallest (lowest) concentration of PEG that results in visible opalescence in the sample well or the opalescence of sample wells in the plate can be read using an automated plate reader or other suitable device.

Solubility Assay of Variable Parameters

In some embodiments, the PEG assay for determining relative solubility of a polypeptide is used to assay the relative solubility of a selected polypeptide under different assay conditions, i.e., using different parameters that can affect solubility. This type of assay is useful, for example, to identify parameters under which the solubility of a polypeptide is appropriate for a particular purpose such as storage and use as a clinical compound.

One example of a parameter that can be varied in the assay is buffer composition. Buffers that can be tested include, but are not limited to, succinate, histidine, or phosphate buffers. In some cases, testing relative solubility of a polypeptide in the presence of different buffers is useful for identifying an appropriate buffer for a particular application of the polypeptide.

Density of a solution containing a polypeptide can also affect solubility. Accordingly, a parameter that can be tested using the assay is effect of varying concentrations of a molecule that can affect density or other properties of a solution on solubility. An example of such a molecule is sucrose. Concentrations of sucrose that can be used in the assay are, for example, about 0.5%-10%. Other molecules that are relatively inert and can affect the density of a solution can also be used, for example, dextran or glycerol.

Another parameter that can be assayed for the effect on relative solubility of a polypeptide is varying ionic strength. Non-limiting examples of ionic strength that can be tested include such cations as Na⁺, Ca²⁺, K⁺, Co²⁺, Cu²⁺, Fe²⁺, Mg²⁺, Ni²⁺, Zn⁺, Al³⁺, Fe³⁺, or such anions as Cl⁻¹, NO₃⁻, PO₄³⁻, SO₄²⁻, CO₃²⁻, or C₂H₃O₂⁻ (acetate).

An additional parameter that can be varied in assays of relative solubility is temperature (e.g., from about 0° C. to about 30° C., about 5° C. to about 40° C., about 5° C. to about 37° C., about 15° C. to about 37° C., or about 25° C. to about 37° C.). Another parameter that can be varied and tested in the assay is pH (e.g., from about pH 5.0 to about pH 8.5; about pH 5.5 to about pH 8.0; about pH 5.5 to about 7.5, and about pH 6.0 to about pH 7.5).

Suitable concentrations of polypeptides used in the assay include, without limitation, about 1 mg/mL to about 200 mg/mL.

As used herein, “actual solubility” of a polypeptide refers to the maximum amount of polypeptide that can be dissolved into a solution, the measurement of which takes place in the absence of a volume-exclusion agent such as PEG. Specific conditions are, for example, temperature, buffer, ionic strength, pH, solution density, or a combination thereof.

As used herein, “relative solubility” of a polypeptide refers to the solubility of one polypeptide (generally, a test polypeptide) compared to a second polypeptide or group of polypeptides, or, in some cases, the solubility of a polypeptide under one set of conditions (parameters) compared to the same polypeptide under one or more different conditions. Unlike actual solubility, relative solubility does not have a numerical value, but rather is used to make comparisons, such as with reference polypeptide standards of known solubility or relative solubility of a polypeptide under different conditions such as buffer, ionic strength, pH, solution density, or a combination of variations of such conditions.

As used herein, “apparent solubility” or “predicted solubility” of a polypeptide is the numeric value calculated by extrapolating the curve generated on a graph when log solubility values are plotted against the PEG concentration of a polypeptide sample, the extrapolation being to the axis representing log solubility and representing the data point corresponding to a polypeptide solubility when the PEG concentration of the polypeptide sample is zero.

The apparent solubility value can include a component reflecting the interactions of the polypeptide with itself in solution. This is referred to as an “activity term” and may inflate the apparent solubility value obtained by extrapolating the line taken from volume-exclusion assays, rendering the apparent solubility value inaccurately high. This is generally the case for polypeptides with relatively high solubility, such as albumin, which has a maximum actual solubility of 677 mg/mL based on the packing density of hexagonally close-packed hard spheres. However, in PEG precipitation experiments, that number may appear much higher owing to the inclusion of the activity term in the apparent solubility. The methods disclosed herein for determining relative solubility do not provide an accurate calculation of actual solubility, but do provide methods for comparing the solubility of polypeptides to each other under the same conditions or the same polypeptide to itself under different experimental conditions.

In one example of an application of a relative solubility assay, a polypeptide or polypeptide of unknown solubility is compared to a polypeptide known to have low solubility, e.g., the P5 antibody in the Examples. A protein or polypeptide having solubility similar to a poorly soluble polypeptide will also have low solubility. Such information is useful for determining, e.g., appropriate conditions for applications using such a protein or polypeptide, or can be used to screen out a protein or polypeptide for applications where low solubility is not acceptable. Thus, a relative solubility assay can be used to identify polypeptides that are likely to cause similar solubility problems in large-scale production if the results of the PEG-precipitation method for the two polypeptides are very similar, or if the test polypeptide shows a lower relative solubility than the polypeptide of known low solubility.

Precipitation of Polypeptides

The relative solubility assay disclosed herein includes PEG precipitation of one or more selected (e.g., test) polypeptides (e.g., at least two selected polypeptides, at least three selected polypeptides, at least five selected polypeptides, at least ten selected polypeptides, or more than ten polypeptides). The number of polypeptides that can be tested in a single assay is generally limited by the available format (e.g., multi-well plate or printed grid) and the ability to carry out the steps for the number of polypeptides within an reasonable time. PEG precipitation is carried out by adding a solution of PEG to an aqueous solution containing the selected polypeptide, resulting in a PEG/polypeptide solution; incubating the PEG/polypeptide solution for a time sufficient to permit precipitation of polypeptide in the solution, typically 30-60 minutes. Different times can be used and may be determined empirically using methods that will be apparent to those in the art. The assay components (including the polypeptide and PEG) are typically mixed, e.g., by pipetting or shaking, at room temperature and incubated at the desired temperature until time sufficient for measurement of the precipitate has elapsed, typically about 30-60 minutes. Precipitated polypeptide can be removed (e.g., by centrifugation) and the amount of polypeptide remaining in the supernatant or in the precipitate is determined, and solubility for that polypeptide is calculated. Alternatively, instead of the collecting of precipitate, precipitation is assayed, e.g., by assaying the opalescence (e.g., turbidity) of the PEG/polypeptide solution. In some cases, precipitation is assayed by determining the amount of precipitate collected by centrifugation or determining the amount of protein in the collected precipitate.

Methods of assaying opalescence are known in the art and include, for example, assaying absorbance at a wavelength of 400 nm or higher by UV/visible spectrophotometer, other methods of photo-electric turbidometry (e.g., automated turbidometry), simple visualization by eye, right angle light scattering, or fluorescence. Examples of PEG suitable for use in a relative solubility assay includes, without limitation, PEG-10K, PEG-20K, or within a range of approximately PEG 4-30K. In general, ultrapure PEG is used although other qualities of PEG preparation can be suitable (e.g., chemical grade, commercial grade, or pharmaceutical grade).

Polypeptides

The methods described herein are generally used for testing the relative solubility of polypeptides including polypeptide fragments. However, the method can be used to test the relative solubility of any type of molecule that can be precipitated using PEG. In general, a polypeptide that is tested for relative solubility using the methods described herein is an isolated or purified protein or polypeptide. Such molecules are generally substantially free of cellular material or other contaminating polypeptides from the cell or tissue source from which the protein or polypeptide is derived, or, when the molecule to be tested is chemically synthesized, the sample containing the molecule is substantially free from chemical precursors or other chemicals. The language “substantially free” means preparation of a selected protein or polypeptide having less than about 30%, 20%, 10%, or 5% (by dry weight), of a protein or polypeptide that is not the selected protein or polypeptide (also referred to herein as a “contaminating polypeptide”), or of chemical precursors. When the selected protein or polypeptide is produced by recombinant means, it is also generally substantially free of culture medium, i.e., culture medium represents less than about 20%, less than about 10%, and less than about 5% of the volume of the protein or polypeptide preparation.

“Polypeptide” as used herein means a chain of amino acids regardless of length or post-translational modifications, and includes, for example, proteins, peptides, protein or polypeptide fragments, and conjugated proteins. The term also includes polypeptides that contain non-naturally-occurring amino acids. Polypeptides can be obtained from any source, for example, secreted recombinant polypeptides, polypeptides isolated from natural sources, non-secreted recombinant polypeptides, or synthetic polypeptides. Polypeptide concentrations suitable for use in the assay are from about 0.5 mg/mL to 10 mg/mL, about 10 mg/mL to 100 mg/mL, and about 100 mg/mL to 300 mg/mL. Proteins used in an assay can be denatured or have secondary or tertiary structures (e.g., naturally occurring structure or structure induced during, for example, isolation. If impurities in the sample are substantially less soluble than the peptide of interest, the apparent solubility will be under estimated. In contrast, if the impurities are substantially more soluble than the peptide of interest, the apparent solubility of the peptide of interest will be overestimated.

Determination of Relative Solubility

To determine the relative solubility of a polypeptide or set of polypeptides, the turbidity or other measure of precipitation (such as protein content of a precipitate or of a supernatant following PEG precipitation of a sample) can be plotted against the variable (e.g., PEG concentration, pH, ionic strength, buffer molarity, sucrose concentration, or a combination thereof). For example, the Y-intercept of a selected polypeptide or set of polypeptides is compared to the Y-intercept of one or more polypeptides assayed under the same conditions and the solubilities of the polypeptides are ranked (e.g., less soluble to more soluble), thereby providing a measure of relative solubility. Other methods of determining relative solubility are described herein, and include visual evaluation of opalescence and correlation of such evaluation with relative solubility.

Validation of the Method

The relative solubility assay was validated by comparing the predicted outcomes of changes in experimental parameters such as:
(i) temperature, which increased the solubility of polypeptide(s),
(ii) starting polypeptide concentration, which did not affect the measurements of relative solubility at a concentration range of about 1 mg/mL to about 100 mg/mL,
(iii) pH, which increased solubility as pH decreased from pH 8.0 to pH 6.0,
(iv) ionic strength of buffer, which reduced solubility as ionic strength was increased, and also was compensated for by the addition of salt (NaCl), and
(v) sucrose, which improved solubility, even of polypeptides having relatively low solubility.

All of these results were consistent with findings related to varying parameters and solubility using methods known in the art. Therefore, the relative solubility assay can be used to provide useful information about the solubility of a polypeptide that is consistent with solubility determined by other methods.

Thus, the results of the relative solubility assay disclosed herein are consistent with predicted outcomes when assay conditions are varied, suggesting further that the PEG precipitation method of determining relative solubility is a suitable substitute for actual solubility determinations, which may require tenfold greater amounts of starting polypeptide.

High Throughput Screening (HTS) Using a Relative Solubility Assay

The relative solubility assay described herein can be used in a method for large scale analysis of selected polypeptides by employing a 96-well format or other format designed to accommodate multiple samples (e.g., in wells or printed grids) for simultaneous analysis.

In an example of such an assay, different polypeptides with similar molecular weights (such as different antibodies, which will have the same slope of the line in the solubility graph if the molecular weights are approximately equal) are suspended at the same polypeptide concentration and are mixed with a range of PEG concentrations (e.g., about 1-20%) in a 96-well or other multi-well format such as a slide printed with a hydrophobic grid, incubated for a sufficient time for precipitation to occur, and visually screened for the lowest PEG concentration that precipitates each polypeptide. The lowest PEG concentration is then correlated with the approximate relative solubility of the polypeptide.

The format allows analysis of multiple polypeptide samples relative to one another by determining the approximate concentration of PEG at which a polypeptide begins to precipitate, as assayed by observation of which samples are becoming visibly clouded or opaque (e.g., assaying turbidity). This technique can thus omit the need for centrifugation of the precipitate and obtaining a concentration reading on the supernatant as in other techniques. However, in some cases of the present method, such methods (e.g., centrifugation and concentration readings) can also be used.

To analyze the results of a high-throughput assay for relative solubility (e.g., an assay used to screen a set of polypeptides for relative solubility), turbidity can be visually screened (by examining the opalescence in the sample wells), or alternatively, automate the process using a UV/visible spectrophotometer with measurements in the 400-600 nm range, for example, at 500 nm.

As used herein, the term “opalescence” means detectable turbidity or other visual indication that a polypeptide solution (e.g., a PEG/polypeptide solution) contains a precipitate. In some cases, opalescence is not detectable to the human eye. In such cases, analysis of samples, e.g., the high-throughput screening samples, can be determined using more sensitive methods such as spectrophotometry, e.g., automated spectrophotometry, by using a visible light spectrophotometer or equivalent means for detecting light absorbance of the samples.

### EXAMPLES

The invention is further illustrated by the following examples. The examples are provided for illustrative purposes only. They are not to be construed as limiting the scope or content of the invention in any way.

### Example 1

### General Methodology for Performing PEG-Precipitation of Polypeptides

All PEG used in the experiments described infra was purchased from Fluka Chemical Corp. (Ronkonkoma, N.Y.). Dissolving PEG in buffered solutions was observed to cause a significant change in the measured pH; as much as 1 pH unit with 40% PEG-10K in 20 mM succinate buffer. This pH change could change the slope of the solubility curve by progressively increasing the pH with increasing PEG concentration. Therefore, the pH values of the 40% PEG-10K stock solutions were adjusted after dissolving PEG in a buffer.

Antibody stock solutions were prepared by dialyzing the polypeptide into a selected buffer and diluted to 10 mg/mL with a buffer. Aliquots of the polypeptide solution and 40% PEG-10K solution were added to 1.5 mL Eppendorf tubes to a final volume of 350 μl according to Table 1, and thoroughly mixed.

TABLE 1

| | Vol. of 40% | |
|---|---|---|
| Target | PEG-10K | Vol. of 10 mg/mL |
| % PEG | (μL) | mAb (μL) |
| 2 | 17.5 | 332.5 |
| 3 | 26.25 | 323.75 |
| 4 | 35 | 315 |
| 5 | 43.75 | 306.25 |
| 6 | 52.5 | 297.5 |
| 7 | 61.25 | 288.75 |
| 8 | 70 | 280 |
| 9 | 78.75 | 271.25 |
| 10 | 87.5 | 262.5 |
| 11 | 96.25 | 253.75 |
| 12 | 105 | 245 |
| 13 | 113.75 | 236.25 |
| 14 | 122.5 | 227.5 |

All solutions were allowed to equilibrate at a target temperature for at least 30 minutes. Precipitation was observed to occur at certain polypeptide to PEG ratios. All mixtures were centrifuged to separate the polypeptide precipitate, and the supernatant assayed by ultraviolet and visible spectrophotometry at 280 nm and 320 nm. The temperature of the samples was maintained at 20° C. 0r 0° C. (in an ice water bath) throughout the incubation and centrifugation process. An ice water bath at 0° C. was chosen to reduce temperature fluctuation because of the high heat capacity of water at 0° C. A solubility diagram was plotted and fitted by exponential function using the saturation solubility data in the log linear scale as a function of PEG concentration.

### Example 2

### Assay for Polypeptide-PEG Interactions

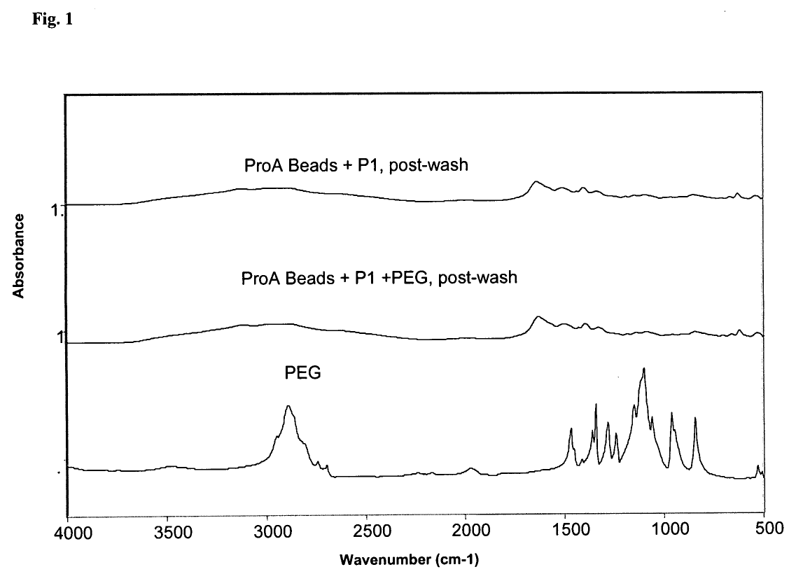

To examine whether polypeptides of interest (selected polypeptides) interacted with PEG, and therefore would interact with PEG in a relative solubility assay, which would adversely affect the analysis of the assay results, a binding study was performed. Two small columns were prepared with 0.5 mL of MabSelect™ ProA resin (GE Healthcare, Piscataway, N.J.) loaded into each column. Both columns were washed with 10 mL 10 mM phosphate pH 7.0 to remove ethanol. Two mL of 30 mg/mL of an antibody (P1) in the same buffer was added to each of the columns, and the flow-through was reloaded onto the column to insure maximum binding. Each column was then washed with 10 mL of binding buffer (10 mM phosphate pH 7.0) to remove unbound polypeptide. Twenty percent PEG-10K in 50 mM histidine pH 6.0 was then added to one of the columns followed by a wash using 10 mL of the same buffer. The resin in each column was suspended in 1 mL of water and each suspension transferred to a 10 mL lyophilization vial. The samples in each of the two vials were lyophilized. The following three samples were analyzed using Fourier Transform Infrared Spectroscopy (FTIR): PEG-10K powder, lyophilized ProA-mAb resin incubated with PEG, and lyophilized ProA-mAb resin not incubated with PEG. Three mg of each powder sample was mixed with 200 mg of KBr, pressed into a 13-mm disk at four tons pressure with a die press. Fourier Transform Infrared Spectroscopy (FTIR) analysis of the KBr pellets was conducted with an MB FTIR spectrometer (ABB Bomen Inc., Quebec, Canada). FTIR is an analytical technique that is used to identify organic materials by measuring the absorption of various infrared light wavelengths by the polypeptide. The absorption of infrared light creates bands of absorption, which are characteristic of specific molecular components and structures. A total of 256 scans at 2 cm⁻¹ resolution were averaged to obtain each spectrum. During data acquisition, the spectrometer was continuously purged with dry air to eliminate the spectral contribution of atmospheric water. As the results in FIG. 1 indicate, PEG does not bind to P1. While conjugated polypeptides are contemplated for testing using a relative solubility assay, a molecule conjugated to a selected polypeptide generally must not interact with PEG. The method described in this Example can be modified using methods known in the art to test for PEG interaction with a molecule.

### Example 3

### Assay for Structural Changes in the Polypeptide

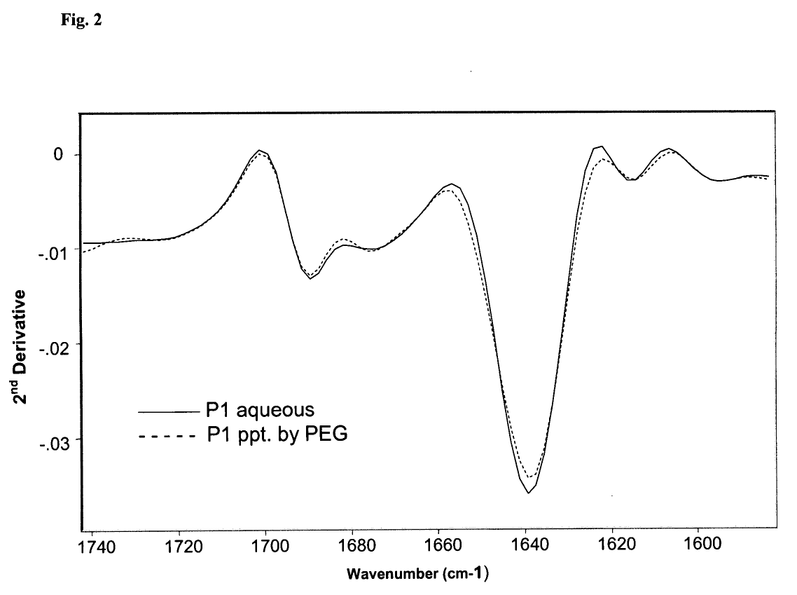

To determine whether any structural changes in the polypeptide takes place during the PEG precipitation protocol, aqueous P1 antibody not contacted with PEG was analyzed in parallel with P1 antibody precipitated by the PEG technique. Ten mg/mL P1 in 50 mM histidine pH 6.0 was precipitated by adding 40% PEG solution to a final PEG concentration of 12%, and the precipitate was collected by centrifugation. The precipitated polypeptide and P1 solution at 30 mg/mL were loaded into a BioCell liquid cell (Biotools, Inc., Wauconda, Ill.) equipped with CaF₂ windows, and measured by ABB Bomen MB FTIR spectrometer. The spectra were corrected for water contribution, smoothed with a 9-point smoothing function, normalized, and analyzed by second derivatization in the amide I region. As shown in FIG. 2, PEG precipitation of the polypeptide did not induce a change in the secondary structure of the polypeptide. This result was consistent with expectations based on knowledge in the art and thus confirms that the PEG precipitation method is useful for determining the relative solubility of a polypeptide.

### Example 4

### Analysis of the Reversibility of the PEG Precipitation Method

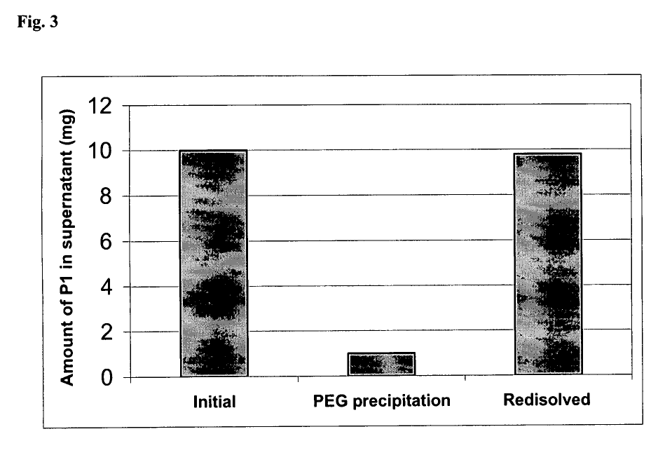

Validation of the PEG precipitation method (relative solubility assay) requires that the volume-exclusion curve generated by measuring polypeptide content in the supernatant following precipitation results from equilibrium between soluble and precipitated polypeptide. Equilibrium indicates that there is no net change between solid and aqueous phases of the polypeptide in the reaction, and depends on the solid phase being capable of returning to the aqueous phase (“reversibility”). To test the reversibility of the disclosed method, PEG-precipitated P1 antibody was re-dissolved and the supernatant was re-quantified to compare with the amount of starting polypeptide. One mL of 10 mg/mL P1 antibody in 50 mM histidine pH 6.0 was precipitated by adding 40% PEG-10K in the same buffer to a final PEG concentration of 14%. Supernatant concentration was measured using a UV-visible spectrophotometer. Two mL of 50 mM histidine pH 6.0 was then added to the mixture to fully dissolve the precipitate, centrifuged, and the concentration of polypeptide in the supernatant was measured. The amount of total soluble polypeptide was calculated by multiplying the concentration and the volume. As shown in the data of FIG. 3, the amount of P1 antibody recovered after being re-dissolved is not significantly less than the starting amount, indicating the method is fully reversible, demonstrating that this assay requirement is met.

### Example 5

### Effect of PEG Molecular Weight

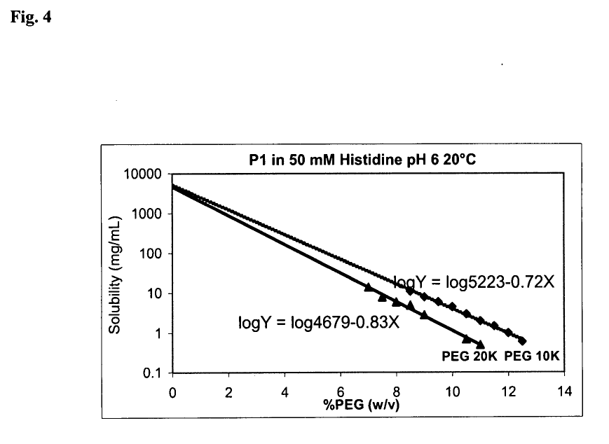

To compare the effectiveness of volume-exclusion with different molecular weights of PEG, solubility was tested using PEG-10K and PEG-20K (FIG. 4). P1 suspended in 50 mM histidine buffer, pH 6.0 was used as a starting polypeptide, and the experiment was carried out at 20° C. Precipitation of 10 mg/mL P1 requires a slightly lower concentration of PEG-20K (about 7% and above) compared to PEG-10K concentration (about 8.5% and above) because PEG-20K has higher efficiency of protein precipitation.

Both types of PEG resulted in a similar Y-intercept despite the difference in the slope, indicating that both PEG types give similar apparent solubility values. The high viscosity of PEG-20K stock solution made it difficult to handle during sample preparation; therefore, PEG-10K was chosen for subsequent studies.

### Example 6

### Effect of Molecular Weight of the Polypeptide

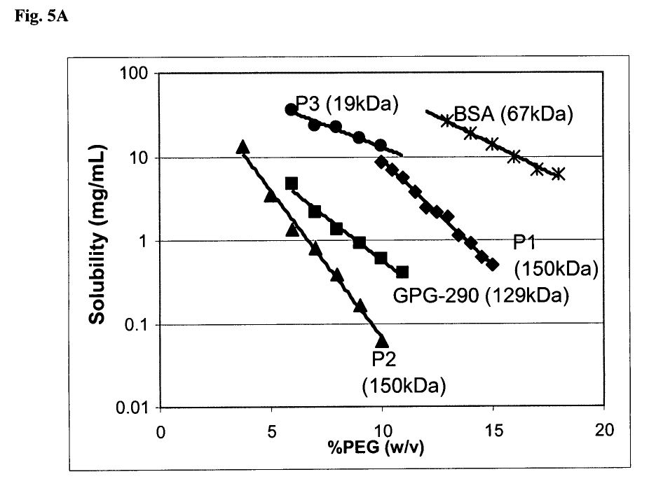

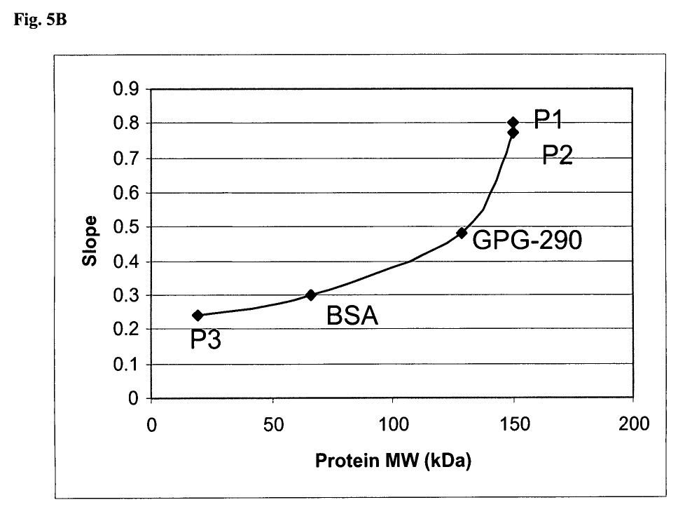

Additional polypeptides with different molecular weights were used to test the effect of polypeptide size on the solubility measurements using the relative solubility assay. FIG. 5A discloses the resulting curves of each polypeptide tested. The slopes of the respective lines for each polypeptide were then plotted against the molecular weight of the polypeptide, and the resulting graph (FIG. 5B) indicates that the slope increases as the polypeptide size increases.

Some noticeable features when using the method of PEG-induced polypeptide precipitation can be understood with reference to FIG. 4. The apparent solubility values of 4679 mg/mL and 5223 mg/mL, which are estimated by the intercept, are inaccurately high. It is reported that the estimated maximum solubility of albumin is 677 mg/mL, i.e., it is sterically impossible to pack much more than 667 mg of protein into 1 mL of volume based on the packing density of hexagonally close-packed hard spheres (Atha and Ingham, J. Biol. Chem. 256:12108-12117 (1981)). Atha and Ingham point out that polypeptides at high concentration result in intercepts that include an activity related term, and therefore exceed the practical solubility limits. Consequently, care should be taken in interpretation of data for highly soluble polypeptides. The extrapolated apparent solubility does not depict the actual solubility. Thus, the PEG precipitation method should be considered qualitative rather than quantitative in the following experiments, i.e., the method can be used to compare one polypeptide to another rather than using the method to determine with accuracy the actual solubility of a single polypeptide.

### Example 7

### Reproducibility of the Relative Solubility Assay

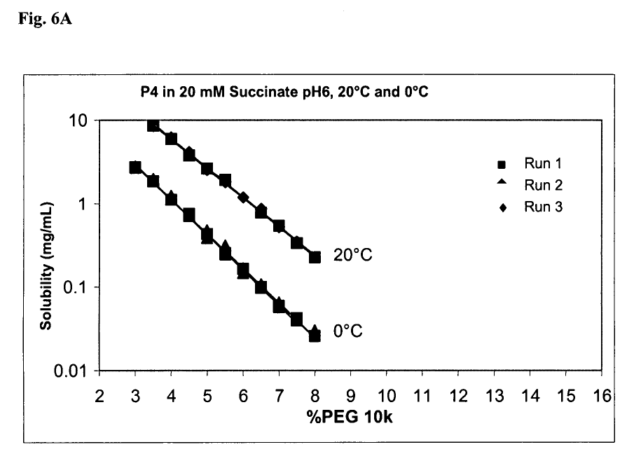

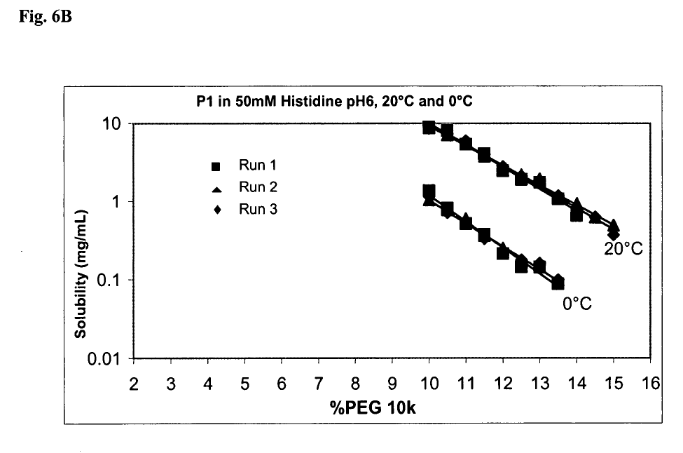

Two different monoclonal antibodies, P4 (in 20 mM succinate pH 6.0) and P1 (in 50 mM histidine pH 6.0) were both used to test the reproducibility of the relative solubility assay using the protocols described in Example 1. Solubility measurements were carried out in triplicate runs on different days (FIGS. 6A and 6B). At both temperatures, good reproducibility of solubility prediction was observed for both monoclonal antibodies. Thus, the method disclosed herein yields reproducible results for polypeptide solubility of the same polypeptide tested multiple times. This reproducibility was also observed when PEG precipitation was carried out at different temperatures (i.e., the initial temperature was tested twice and yielded consistent results, and the second temperature was tested twice and produced consistent results). These results indicate that there is no significant inter-assay variability in solubility determinations using the PEG precipitation method. This feature is important for an assay such as the relative solubility assay that is intended for, e.g., commercial use.

### Example 8

### Effect of Starting Polypeptide Concentration

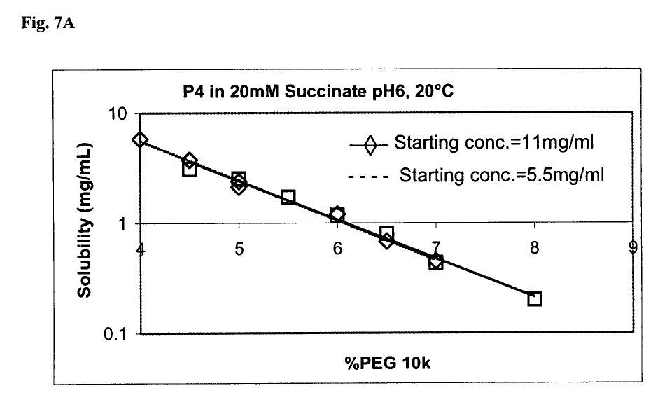

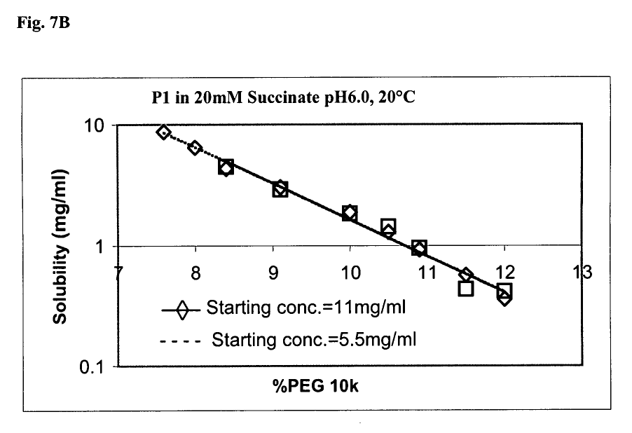

To determine whether the PEG-precipitation method described here remained independent of polypeptide concentration, P4 antibody and P1 antibody were both tested at a low concentration of 5.5 mg/mL and a high concentration of 11 mg/mL using the protocol of Example 1. The effect of varying the total polypeptide content of the solution on the predicted solubility of the polypeptide is illustrated in FIGS. 7A and 7B. For both tested antibodies, the extrapolated solubility values are independent of the total polypeptide concentration between 5.5 mg/mL and 11 mg/mL.

These data demonstrate that the PEG precipitation method for determining solubility can be used over a range of protein concentrations.

### Example 9

### Effect of Temperature

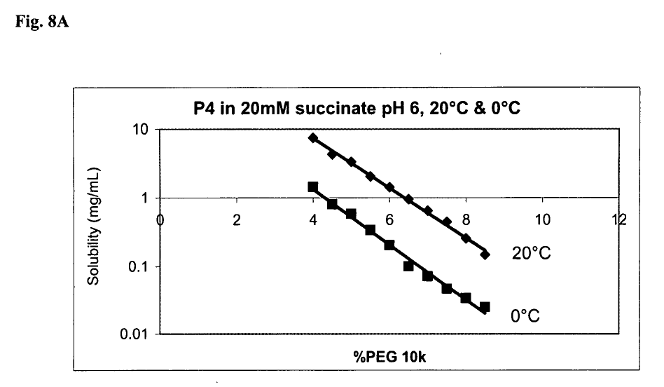

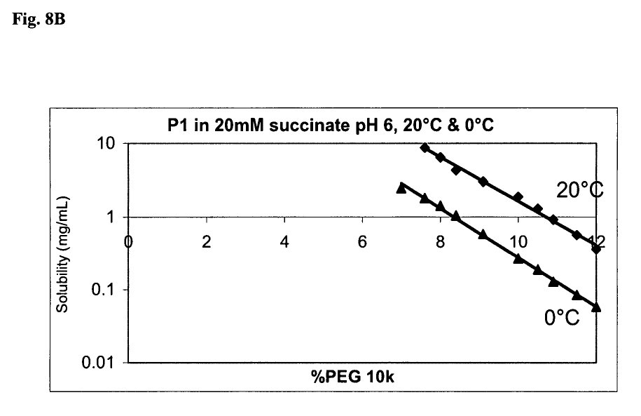

To test whether the PEG-precipitation method for determining relative solubility accords with the known effect of temperature on solubility of polypeptides, P4 and P1 were both tested using the general protocol of Example 1 but at two different temperatures: 0° C. and 20° C. Increased apparent solubility was found at elevated temperature (FIGS. 8A and 8B) using this approach. A similar temperature effect on solubility has been found empirically through experimentation, e.g., using a method testing actual solubility. Thus, the PEG precipitation method described herein is consistent with the results expected using methods testing actual solubility.

### Example 10

### Effect of pH

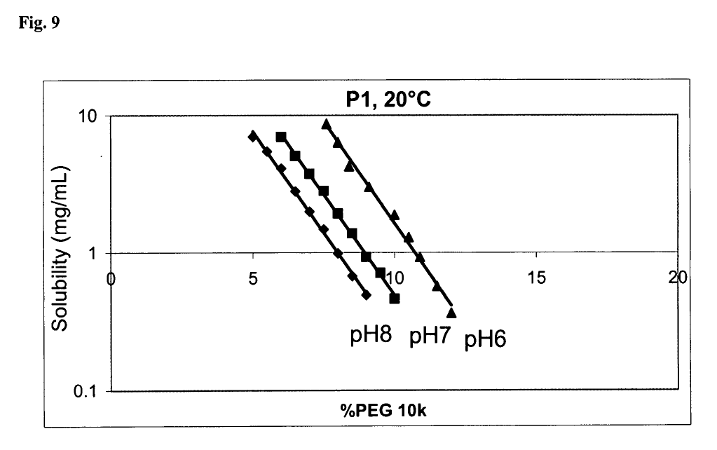

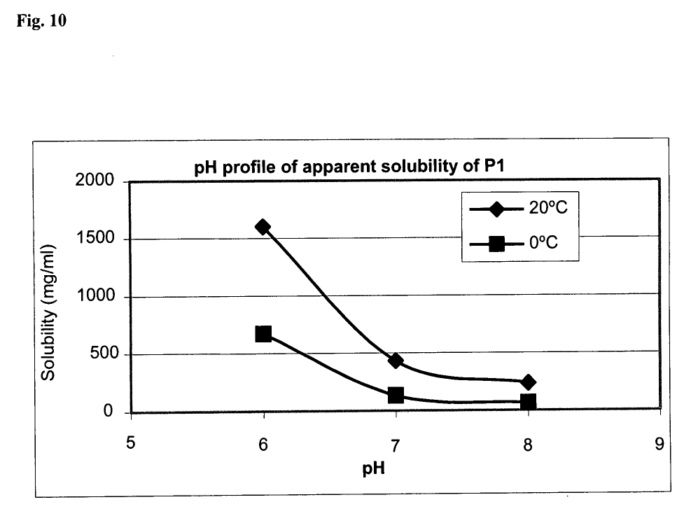

The solubility of P1 at various pHs was tested (FIG. 9). The log-linear response of P1 concentration versus percent PEG concentration shows that the Y-intercept (zero PEG concentration, i.e., apparent solubility) decreases as pH increases from pH 6 to pH 8, but the slopes are not different. The pH profile (FIG. 10) correlates well with the expectation that a polypeptide has lowest solubility at pH around its pI (7.5-8.0 for P1).

These data further demonstrate that the PEG precipitation method can produce results consistent with other methods, such as those for determining actual solubility.

### Example 11

### Effect of Buffer and Ionic Strength

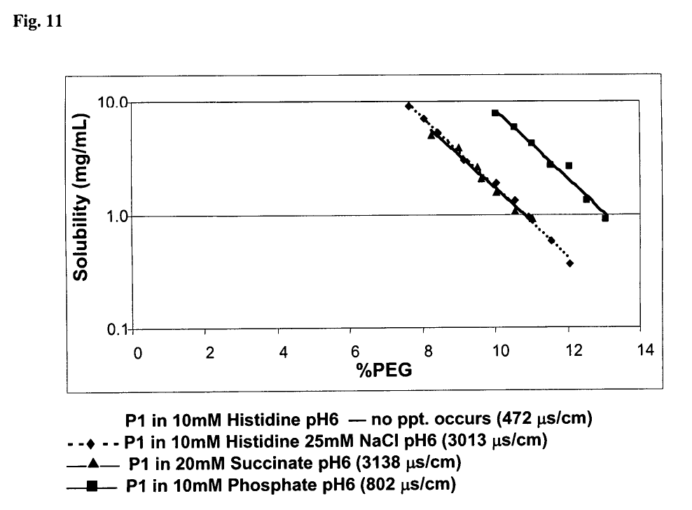

Apparent solubility values for P1 antibody were tested at pH 6.0 using different buffers such as succinate, histidine, and phosphate, and different results were obtained with various buffers (FIG. 11). These data demonstrate that the low ionic strength of 10 mM histidine buffer is the explanation for the lack of precipitation that occurred for 10 mg/mL P1 in that buffer, which could subsequently be compensated for by the addition of NaCl. Therefore, when performing a relative solubility assay, an increase in ionic strength can decrease the solubility of a protein. This is in accordance with expected measurements of actual solubility. This further validates the method as concurring with results obtained with standard solubility assays known in the art.

### Example 12

### Effect of Sucrose

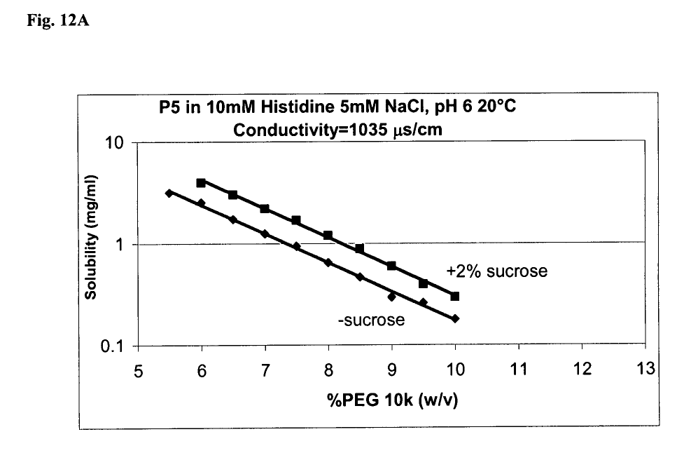

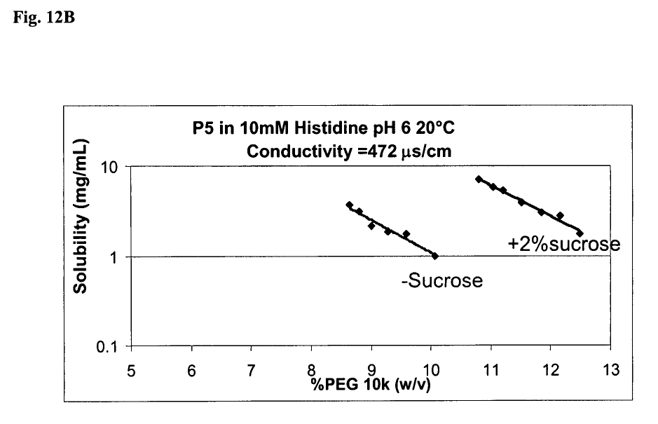

Previous studies have shown that sucrose enhances solubility of P5 during ultrafiltration/diafiltration. To confirm the reliability of the relative solubility assay method, the effect of sucrose on predicted solubility of P5 was tested (FIGS. 12A and 12B). P5 in 10 mM histidine buffer at pH 6.0, 20° C. was compared with or without the addition of 2% sucrose. The results of these experiments are shown in FIG. 12B, and indicate that the predicted solubility of P5 increased with the presence of sucrose in both buffers tested.

The magnitude of sucrose-induced solubility enhancement is generally higher in buffer with low ionic strength. This was tested in a relative solubility assay by adding 5 mM NaCl to both the sucrose and non-sucrose samples. As indicated in FIG. 12A, NaCl greatly decreased the sucrose-induced improvement in solubility. These results of a relative solubility assay agree well with the previous experimentally determined effect of sucrose on solubility, further validating the relative solubility approach.

### Example 13

### Employing a Relative Solubility Assay in High Throughput Screening (HTS)

A 96-well plate format for high throughput screening was used in a demonstration of an application of a relative solubility assay in a higher throughput format using a selection of monoclonal antibodies. Because the slope of the phase diagram remained constant for different monoclonal antibodies under all different conditions (buffer, temperature, concentration) tested above, a simplified version of HTS was designed for this study. All monoclonal antibodies were dialyzed in 50 mM histidine pH 6.0 and their concentrations were adjusted to 10 mg/mL. Forty percent PEG-10K stock solution was prepared in the same buffer and pH was adjusted to 6.0. A quartz 96-well plate was prepared by filling wells with different ratios of monoclonal antibody to PEG-10K stock solution according to Table 2 to give a final volume of 200 μl in each well. Each row was designated for a specific monoclonal with increased final PEG concentration from 2% in column #1 to 16% in column #12. All samples were mixed by pipetting up and down five times, followed by incubation at room temperature for 15 minutes.

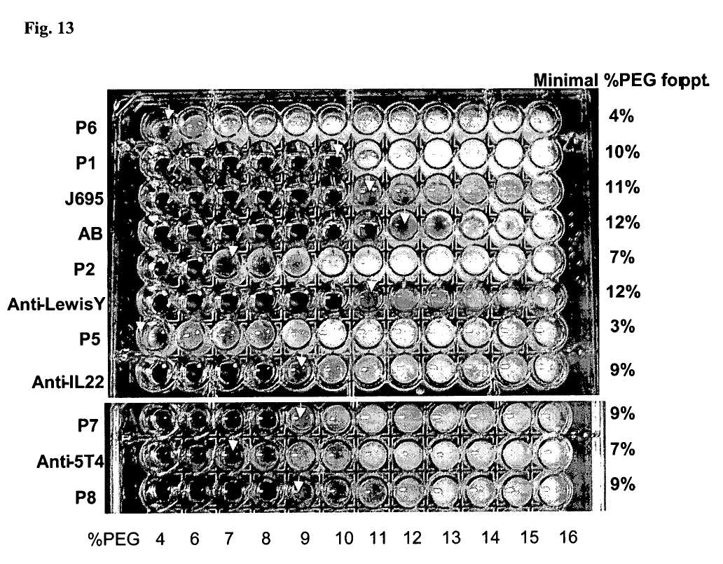

When the initial polypeptide concentrations of all monoclonal were adjusted to the same level, more soluble monoclonal antibodies required a higher percentage of PEG to precipate. Therefore, the minimum percentage of PEG needed for polypeptide precipitation indicates relative solubility of the polypeptide (FIG. 13). This simplified version of the method avoids centrifugation, dilution and concentration measurement of the supernatant following the precipitation step, resulting in high efficiency and reduced need for polypeptide material.

TABLE 2

| | Vol. of | Vol. of |
|---|---|---|
| | 40% | 10 mg/mL |
| Target | PEG-10K | mAb |
| % PEG | (μL) | (μL) |
| 2 | 10 | 190 |
| 4 | 20 | 180 |
| 5 | 25 | 175 |
| 6 | 30 | 170 |
| 7 | 35 | 165 |
| 8 | 40 | 160 |
| 9 | 45 | 155 |
| 10 | 50 | 150 |
| 11 | 55 | 145 |
| 12 | 60 | 140 |
| 13 | 65 | 135 |
| 14 | 70 | 130 |
| 15 | 75 | 125 |
| 16 | 80 | 120 |

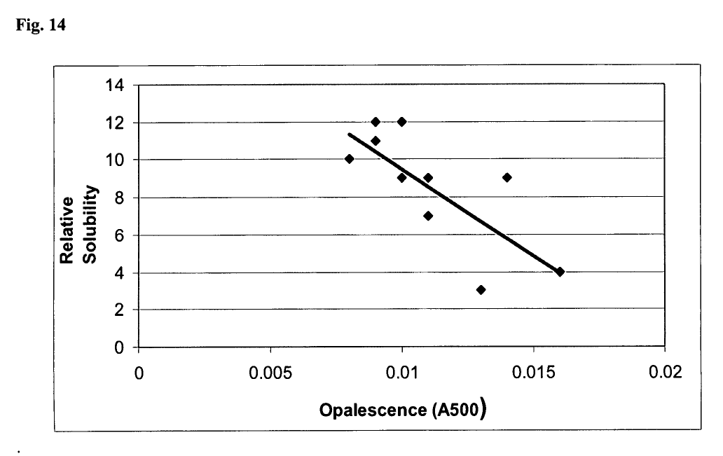

The relationship of the opalescence of the samples (indicating precipitation) of monoclonal antibodies at 90 mg/mL was measured by spectrophotometer absorbance on a SPECTRAmax Plus384 Microplate Spectrophotometer (Molecular Devices Corp., Sunnyvale, Calif.) at 500 nm (A₅₀₀), with the resulting relationship with the relative solubility (i.e., the lowest PEG concentration at which precipitation was observed) plotted on the graph in FIG. 14. These results indicate that the opalescence increases as the relative solubility decreases.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A method for predicting the relative solubility of a test polypeptide, the method comprising;
a. providing one or more samples of a test polypeptide in a solution, thereby providing test samples;
b. contacting the test samples with different concentrations of polyethylene glycol (PEG), thereby forming a precipitated sample;
c. determining the precipitation of each test sample contacted with PEG; and
d. correlating the amount of precipitation of the test polypeptide in the precipitated sample with solubility of at least one reference polypeptide sample analyzed under corresponding conditions, thereby determining the solubility of the test polypeptide relative to the reference polypeptide sample; or correlating the amount of precipitation of the test polypeptide in the precipitated sample under different experimental conditions, thereby determining the relative solubility of the test polypeptide under each experimental condition.

2. The method of claim 1, wherein the test polypeptide is an antibody.

3. The method of claim 1, wherein the test polypeptide is a molecule that can bind to a ligand.

4. The method of claim 1, wherein the test polypeptide is a soluble receptor.

5. The method of claim 1, wherein the test polypeptide is an antibody fragment.

6. The method of claim 1, further comprising graphing the log of the solubility values determined for each sample against the PEG concentration of that sample and extrapolating the resulting line to zero percent PEG, thereby providing an apparent solubility value for the polypeptide.

7. The method of claim 1, wherein the test polypeptide does not bind to PEG.

8. The method of claim 1, wherein the PEG precipitation is reversible.

9. The method of claim 1, wherein the PEG does not change the secondary structure of the test polypeptide.

10. The method of claim 1, wherein the starting concentration of the test polypeptide to be analyzed does not substantially affect the resulting solubility value.

11. The method of claim 1, wherein increasing the temperature increases the solubility value for a selected PEG concentration.

12. The method of claim 1, wherein the addition of sucrose to the buffer increases the solubility of the test polypeptide.

13. The method of claim 1, wherein the slope of the curve resulting from plotting the log solubility values of a higher molecular weight polypeptide sample against the PEG concentration increases relative to the slope of the curve of a lower molecular weight polypeptide.

14. The method of claim 1, wherein the reference is a polypeptide of known solubility.

15. The method of claim 1, wherein precipitation is assayed by determining turbidity.

16. The method of claim 1, wherein the precipitated sample is centrifuged and the amount of precipitate is determined, the amount of protein in the supernatant is determined, or the amount of protein in the precipitate is determined.

17. A method for determining the relative solubility of a polypeptide compared to at least one other polypeptide of approximately the same molecular weight, the method comprising:
a. providing a sample of at least two different polypeptides at the same concentration;
b. contacting each polypeptide sample with a range of test PEG concentrations;
c. determining the lowest test PEG concentration that precipitates a polypeptide sample, thereby determining a minimum percentage of PEG that precipitates each polypeptide; and
d. correlating the minimum percentage of PEG with the solubility of each polypeptide relative to each other polypeptide.

18. The method of 17, wherein at least (b) to (c) are performed in a 96-well plate format.

19. The method of 17, wherein the range of PEG concentrations is about 2%-16%.

20. The method of 17, wherein the plate is read visually by determining the smallest test concentration of PEG that causes opalescence of a sample.

21. The method of 17, wherein the opalescence of samples in the plate is read using an automated plate reader.
